(19) United States
(12) Patent Application Publication
(10) Pub. No.: US 2010/0069687 A1
(43) Pub. Date: Mar. 18, 2010

(54) PROCESS FOR SYNTHESIS OF POLYALPHAOLEFIN AND REMOVAL OF RESIDUAL CATALYST COMPONENTS

(75) Inventors: Vilen KOSOVER, Cheshire, CT (US); Jesus R. FABIAN, Wethersfield, CT (US); Daniel C. KNOWLES, Southbury, CT (US); Mitchel COHN, West Haven, CT (US); Werner A. THURING, Ontario (CA)

Correspondence Address: Patent Administrator;Chemtura Corporation, 199 Benson Road, Middlebury, CT, 06749, US

(73) Assignee: Chemtura Corporation, Middlebury, CT (US)

(21) Appl. No.: 12/577,580

(22) Filed: Oct. 12, 2009

Related U.S. Application Data
(63) Continuation-in-part of application No. 12/549,559, filed on Aug. 28, 2009.
Application No. 12/549,559 is a continuation-in-part of application No. 11/516,452, filed on Sep. 6, 2006, now Pat. No. 7,601,255.

(51) Int. Cl. C07C 2/04; C07C 2/08; C07C 7/20

(52) U.S. Cl. 585/1; 585/521; 585/522; 585/520

(57) ABSTRACT

A process for reducing the level of residual catalyst comprising one or more 1-halo-2-methylpropanes and a Group 13 metal catalyst from a crude polyolefins product, the process comprising contacting the crude organic product with a solid adsorbent in an adsorbent system. Also provided is a co-catalyst system for polymerizing alpha olefins.

### CROSS REFERENCE TO RELATED APPLICATIONS

This application is a continuation-in-part application of U.S. application Ser. No. 12/549,559, filed Aug. 28, 2009, and U.S. application Ser. No. 11/516,452, filed Sep. 6, 2006, both of which are incorporated herein by reference in their entirety.

### FIELD OF THE INVENTION

The present invention generally relates to processes for synthesizing polyalphaolefins in the presence of a Group 13 metal catalyst and a 1-halo-2-methylpropane catalyst system. The present invention also relates to processes for removing residual catalyst components, i.e. metals and halides, from crude polyalphaolefin product.

### BACKGROUND OF THE INVENTION

In organic synthesis, catalysts are very often soluble in the resulting crude organic product and cannot be removed by simple filtration. These catalysts and co-catalysts contain at least one alkylhalide, alkoxyhalide, metal halide, metal oxyhalide, alkyl metal, alkoxy metal, boron compound and coordinated metal compound used alone or in any combination, Friedel-Crafts catalyst, and supported or unsupported metallocene catalyst. One conventional catalyst system, described in U.S. Pat. No. 4,469,910, comprises 2,3-dibromo butane and triethylaluminum (TEA). This catalyst system contains a relatively high bromine concentration and thus increases the halogen content in the resulting crude polyalphaolefin (PAO) product.

Following the polymerization of an alpha olefin, the crude PAO product will contain dissolved catalyst, including halides and metals of the catalysts, which needs to be removed prior to finishing, e.g., hydrogenation. Accordingly, in finishing PAO, considerable amounts of money are spent on hydrogenation catalyst and hydrogen usage. Much of this cost is a direct result of the high residual polymerization catalyst levels remaining in the unfinished or crude product, since the residual metal and halogen from the polymerization catalyst render higher hydrogenation catalyst loadings necessary during hydrogenation of the crude PAO product due to the hydrogenation catalyst being poisoned by the halogen.

The insufficient removal of catalysts, e.g., olefin polymerization catalysts, and, in particular, their metallic and halogen components, from a liquid organic product such as liquid olefin polymer also results in other undesirable problems. For example, the presence of catalyst residues may cause discoloration of the resulting polymerization products, the generation of hydrogen halide gas owing to the thermal degradation of the catalyst, the degradation or decomposition of the organic compounds owing to structural change during subsequent distillation, the poisoning by halogen contaminants of hydrogenation catalysts during subsequent polymer treatment, the formation of aluminum hydroxide slimes which are difficult to handle and the like.

Efforts have been made to remove olefin polymerization catalysts from the liquid olefin polymer. For example, U.S. Pat. No. 4,028,485 discloses a process for removing hydrogenation catalyst residue from solutions of hydrogenated olefins or olefinic polymers containing them comprising treating such solutions with a non-aqueous acid followed by neutralization with an anhydrous base and filtration. U.S. Pat. No. 4,122,126 discloses a method for removing an aluminum halide or its complex catalyst from a polymerization product comprising the steps of adding to the polymerization product an aprotic polar solvent in an amount of 1 through 6 mol per one mol of the aluminum halide in the catalyst present in the product and sufficiently mixing them at a temperature of 70° C. through 150° C., and then filtering the mixture at a temperature of 70° C. through 150° C. The addition of the aprotic polar solvent facilitates the separation of the catalyst from the polymerization product.

U.S. Pat. No. 4,476,297 discloses that the content of titanium and light metal halides and aluminum compounds in polyolefins emanating from the catalyst system can be considerably reduced by treatment with a higher, preferably branched, aliphatic monocarboxylic acid having 6 to 10 carbon atoms.

U.S. Pat. No. 4,642,408 discloses the removal of nickel, aluminum and chlorine derivatives, which remain dissolved in olefin oligomers after oligomerization in the presence of a catalyst containing such derivatives by treatment with oxygen or a gas containing oxygen, anhydrous ammonia, and a solution of an alkali metal hydroxide.

U.S. Pat. No. 4,701,489 discloses that the catalyst residues present in an on-purpose produced amorphous polyalphaolefin are deactivated by contacting the molten polymer with sufficient water to provide at least a 3:1 water/Al mole ratio and then the polymer is stabilized with a hindered phenolic antioxidant.

U.S. Pat. No. 7,473,815 discloses a method for reducing levels of residual halogen and Group IIIb metals in a crude PAO products in the presence of a catalyst comprising the halogen and Group IIIb metals, wherein the method comprises: (a) washing the crude poly(alpha-olefin) with water; (b) separating the aqueous and organic phases; (c) adding an adsorbent selected from the group consisting of magnesium silicates, calcium silicates, aluminum silicates, aluminum oxides, and clays to the organic phase to form a slurry; (d) heating the slurry under reduced pressure at a temperature of at least about 180° C. for at least about thirty minutes; and then (e) separating the adsorbent from the slurry. However, this water washing method is overly complicated, employs additional steps, e.g., decantation, filtration and drying, and produces a large amount of aqueous waste. It is also difficult to run on a continuous basis.

It would be desirable to provide an improved process for synthesizing polyalphaolefin and removing the catalyst residues from polyalphaolefins, as fully as possible prior to subsequent treatment and/or use of such products.

### SUMMARY OF THE INVENTION

In accordance with an embodiment of the present invention, the invention is to a process for synthesizing polyalphaolefin comprising polymerizing an alpha olefin monomer in the presence of a co-catalyst system under polymerization conditions, wherein the co-catalyst system comprises a Group 13 metal catalyst and a 1-halo-2-methylpropane. Preferably, the 1-halo-2-methylpropane is selected from the group consisting of 1-chloro-2-methylpropane, 1-bromo-2-methylpropane, or 1-iodo-2-methylpropane. In one embodiment, the Group 13 metal catalyst is an alkyl-aluminum compound selected from the group consisting of trimethylaluminum, triethylaluminum, diethyl(propyl)aluminum, diethyl(butyl)aluminum, ethyl(dipropyl)aluminum, ethyl(dibutyl)aluminum tripropylaluminum, triisopropylaluminum, and tributylaluminum.

In another embodiment, the invention is to a process comprising the steps of reducing a residual level of the co-catalyst system used to form the polyalphaolefin from the polyalphaolefin by contacting the polyalphaolefin with a treatment comprising a solid adsorbent selected from the group consisting of an oxide or hydroxide of magnesium, calcium, strontium, barium, sodium and potassium; and filtering the polyalphaolefin to remove a metal of the co-catalyst system.

### DETAILED DESCRIPTION OF THE PREFERRED EMBODIMENTS

The present invention generally relates to synthesizing polyalphaolefin (PAO) using a co-catalyst system. The co-catalyst system includes a Group 13 metal catalyst and 1-halo-2-methylpropane. Surprisingly and unexpectedly, the co-catalyst system polymerizes alpha-olefin monomer to form PAO having higher viscosity at lower halogen levels compared to other alkyl halides. This allows the use of comparatively less catalysts while increasing the production of PAO. In addition, use less catalysts reduces the amount of residual catalyst to be removed from the crude PAO product.

In one embodiment, the PAO has a kinematic viscosity at 100° C. that is greater than 95 cSt, e.g., greater than 100 cSt or greater than 120 cSt. In terms of ranges, the kinematic viscosity at 100° C. is from 95 to 3,000 cSt, e.g., from 100 to 1500 cSt or from 120 to 600 cSt. In one exemplary embodiment, when it is desirable to produce 100 cSt PAO, the PAO product produced by the catalyst system of the present invention may be greater than 100 cSt. This PAO product may then be blended with another PAO having a viscosity of less than 100 cSt.

In one embodiment, molar ratio of the halide in the 1-halo-2-methylpropane to the metal in the Group 13 metal catalyst is from 2:1 to 16:1, e.g., from 2.5:1 to 5:1 or from 3:1 to 4.5:1. The concentration of Group 13 metal catalyst present during polymerization is from 0.1 to 10.0 wt %, e.g., from 0.8 to 2.5 wt %, or from 0.9 to 2.3 wt %, based on the total weight of reactants. The concentration for Group 13 metal catalyst is provided for the neat catalyst. In some embodiments the Group 13 metal catalyst is diluted in from 10-90 wt % of alpha olefin monomer, e.g., 15-80 wt % or from 25-75 wt %. The concentration of 1-halo-2-methylpropane present during polymerization is from 0.5 to 6.0 wt %, e.g., from 1.5 to 4.0 wt %, or from 2.6 to 2.9 wt %, based on the total weight of reactants. In one embodiment, the total concentration of co-catalyst is from 0.15 to 10 wt %, e.g., from 0.25 to 6 wt %, or from 0.35 to 2 wt %, based on the weight of the alpha olefin present. In one embodiment, the corresponding level of halide, i.e. bromide, of the catalyst is less than 2.3 wt %, e.g., less than 1.9 wt %, or less than 1.7 wt %.

Each compound may be added separately to the polymerization reactor and the co-catalyst system may be formed in situ.

The Group 13 metal catalyst includes those having the structure:

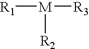

wherein M is a Group 13 metal selected from the group consisting of boron, aluminum, gallium, indium and thallium and R₁, R₂ and R₃ are independently selected from the group consisting of hydrogen, linear and branched C₁-C₁₀ alkyl groups, linear or branched C₂-C₁₀ alkenyl groups, and substituted or unsubstituted C₅-C₁₀ cycloalkyl groups, provided that at least one of R₁, R₂ and R₃ is not hydrogen and more preferably none of R₁, R₂ and R₃ is hydrogen. In one embodiment, R₁, R₂ and R₃ are independently selected from the group consisting of linear and branched C₁-C₁₀ alkyl groups, e.g., linear and branched C₂-C₆ alkyl groups or linear and branched C₂-C₄ alkyl groups. Preferred Group 13 metal catalysts include alkyl-aluminum compounds such as trimethylaluminum, triethylaluminum, diethyl(propyl)aluminum, diethyl(butyl)aluminum, ethyl(dipropyl)aluminum, ethyl(dibutyl)aluminum tripropylaluminum, triisopropylaluminum, and tributylaluminum. Most preferably triethylaluminum (TEA) is used in the co-catalyst systems of the present invention.

The 1-halo-2-methylpropane is selected from the group consisting of 1-chloro-2-methylpropane, 1-bromo-2-methylpropane, or 1-iodo-2-methylpropane. Most preferably the 1-halo-2-methylpropane is 1-bromo-2-methylpropane, also referred to isobutyl bromide (IBB). Preferably, the co-catalyst systems of the present invention do not include any further alkyl halide or halide compounds.

In one embodiment the co-catalyst systems are substantially free of and, more preferably, do not include any other alkali metals, alkaline metals or Group 3-12 metals, such as chromium.

As used herein, the term alpha-olefin monomer or alpha olefin means a linear or branched monoolefin in which the double bond thereof is at the alpha position of the carbon chain of the monoolefin. The alpha olefins suitable for use in the preparation of the polyalphaolefin polymerization products described herein can contain from 2 to 20 carbon atoms, e.g., from 3 to 12 carbon atoms or from 6 to 10 carbon atoms. Examples of such alpha olefins include, but are not limited to, ethylene, propylene, 2-methylpropene, 1-butene, 3-methyl-1-butene, 1-pentene, 4-methyl-1-pentene, 1-hexene, 1-heptene, 1-octene, 1-nonene, 1-decene, 1-undecene, 1-dodecene, 1-tridecene, 1-tetradecene, 1-pentadecene, 1-hexadecene, 1-heptadecene, 1-octadecene, 1-nonadecene, 1-eicosene and the like and vinyl aromatic monomers such as styrene, α-methyl styrene and the like and mixtures thereof. An alpha olefin used in the manufacture of the polyalphaolefin polymerization products of the inventive process can contain substantially one type, i.e., number of carbon atoms per molecule, of alpha olefin or it can be a mixture of two or more types of alpha olefins.

Alpha olefins are polymerized in the presence of the co-catalyst of the invention, preferably at temperature of from 0 to 200° C., e.g., from 30 to 180° C. or from 25 to 45° C., and under pressures of about 1 atm. The polymerization may be conducted in an inert atmosphere, such as a nitrogen atmosphere. Preferably, the polymerization reaction is conducted substantially in the absence of moisture and/or air. The polymerization may be conducted in a continuous reactor having a residence time, for example, of from 0.1 to 20 hours, e.g., from 0.5 to 10 hours or from 1 to 10 hours. Higher residence times may be preferred for commercial production of PAO. Varying these polymerization conditions may vary the viscosity of the resulting polymer. While, increasing residence time tends to increase viscosity, however, in one embodiment, it is desirable to reduce residence time to increase production of PAO.

Exemplary PAO products formed by the systems and processes of the invention are homopolymers and include, but are not limited to, polyethylene, polypropylene, poly(2-methylpropene), polybutene, poly(3-methyl-1-butene), polypentene, poly(4-methyl-1-pentene), polyhexene, polyheptene, polyoctene, polynonene, poly(3-methyl-1-nonene), polydecene, polyundecene, polydodecene, polytridecene, polytetradecene, polypentadecene, polyhexadecene, polyheptadecene, polyoctadecene, polynonadecene, and polyeicosene. Co-polymers may also be formed by the inventive processes where co-monomer, for example, is fed together to the reaction system.

In addition, the present invention is directed to a process for reducing the level of residual catalyst employed in the polymerization of alpha olefin. Preferably the residual Group 13 metal is reduced to an amount of less than 100 wppm, e.g., less than 25 wppm or less than 10 wppm. Preferably the residual halide is reduced to an amount of less than 3000 wppm, e.g., less than 1500 wppm or less than 500 wppm.

In one embodiment, the crude PAO product containing residual catalyst is contacted with a solid adsorbent in an adsorbent system to reduce the level of the residual catalyst. Preferably, there is no washing step following the step of contacting the crude PAO product with the solid adsorbent. Suitable adsorbents include, but are not limited to, basic materials, e.g., a basic compound of an alkaline earth metal, acidic materials, e.g., silica gel, and the like and mixtures thereof. Useful basic compounds of alkaline earth metal include oxides, hydroxides, carbonates, bicarbonates or a mixture thereof of magnesium, calcium, strontium or barium and most preferably calcium. Preferred basic compounds include calcium oxide or calcium hydroxide (e.g., quick lime or slaked lime).

The adsorption may be achieved by mixing the liquid crude PAO product with an absorbent in certain proportions and subsequently removing the adsorbent by separation (e.g. filtration, centrifugation or settling) or by passing the liquid crude PAO product through a fixed bed, e.g., a packed column, containing the adsorbent. A suitable filter can be any pressure filter or vacuum filter of suitable porosity to separate the adsorbent. A suitable column can be a column sized to give adequate residence time and velocity for the adsorption to take place packed with adsorbent. If desired, when using a filter, a filter aid, e.g., diatomaceous earth, may be employed to expedite the filtering of the crude PAO product. Generally, the amount of adsorbent used in the adsorbent system can vary widely depending on the amount of liquid crude organic product used in the process and can readily be determined by one skilled in the art. The temperature of adsorption preferably is from room temperature to about 150° C., and preferably about 40° C. to about 60° C.; the residence time preferably is from about 1 minute to about 60 minutes, and more preferably from about 15 minutes to about 30 minutes. The amount of adsorbent may be at least about 1.1 mole for about 1 mole of catalyst.

In one embodiment, there is no shortstopping with water or low molecular weight alcohols to remove residual catalyst. The methods of the present invention may achieve low levels of residual catalyst without further washing, filtration or distillation.

The process of the present invention to remove residual catalyst is advantageously shortened by avoiding the use of a water washing step, a decantation step and a drying step. Further, the processes of the present invention may be run continuously and produce only solid waste which is relatively non-hazardous.

The following non-limiting examples are illustrative of the present invention.

### Example 1

1-decene was polymerized in a continuous reactor. The reaction temperature was 40° C. and the residence time was 2.8 hours. The combined feed rate of the pre-diluted TEA in decene and pre-diluted alkyl halide in decene is 6.0 g/min. The TEA used is a solution containing 25 wt % of TEA and 75% decene. The molar ratio of bromide to aluminum was 3.3:1 for each of the runs 1-37 in Table 1 using the weight amounts shown in Table 1. Grams are provided in Table 1 based on 100 grams of decene. Table 1 reports the viscosities of the obtained PAO products.

TABLE 1

| | | Alkyl Halide | | | Kinematic |
|---|---|---|---|---|---|
| | TEA | | Weight | Bromide | Viscosity @ |
| Run | (grams) | Type | (grams) | (grams) | 100° C. (cSt) |
| 1 | 0.98 | 1-bromo-2-methyl-propane (IBB) | 1.31 | 0.76 | 35.1 |
| 2 | 1.96 | IBB | 2.62 | 1.53 | 85.8 |
| 3 | 2.11 | IBB | 2.83 | 1.65 | 98.7 |
| 4 | 2.13 | IBB | 2.85 | 1.66 | 108.0 |
| 5 | 2.25 | IBB | 3 | 1.75 | 118.5 |
| 6 | 2.95 | IBB | 3.94 | 2.30 | 1575.7 |
| 7 | 2.95 | 2,3-dibromo-butane (DBB) | 3.1 | 2.29 | 102.0 |
| 8 | 2.95 | DBB | 3.1 | 2.29 | 101.6 |
| 9 | 2.95 | DBB | 3.1 | 2.29 | 139.0 |
| 10 | 0.98 | t-butyl bromide | 1.33 | 0.78 | 37.2 |
| 11 | 1.96 | t-butyl bromide | 2.66 | 1.55 | 90.3 |
| 12 | 2 | t-butyl bromide | 2.7 | 1.57 | 87.6 |
| 13 | 2.22 | t-butyl bromide | 3 | 1.75 | 107.5 |
| 14 | 2.22 | t-butyl bromide | 3 | 1.75 | 86.7 |
| 15 | 2.22 | t-butyl bromide | 3 | 1.75 | 102.0 |
| 16 | 2.22 | t-butyl bromide | 3 | 1.75 | 106.8 |
| 17 | 2.22 | t-butyl bromide | 3 | 1.75 | 95.0 |
| 18 | 2.22 | t-butyl bromide | 3 | 1.75 | 86.6 |
| 19 | 2.22 | t-butyl bromide | 3 | 1.75 | 99.6 |
| 20 | 2.26 | t-butyl bromide | 3.05 | 1.78 | 90.8 |
| 21 | 2.26 | t-butyl bromide | 3.05 | 1.78 | 92.6 |
| 22 | 2.29 | t-butyl bromide | 3.1 | 1.81 | 98.8 |
| 23 | 2.29 | t-butyl bromide | 3.1 | 1.81 | 101.8 |
| 24 | 2.41 | t-butyl bromide | 3.25 | 1.89 | 118.9 |
| 25 | 2.95 | t-butyl bromide | 3.98 | 2.32 | 552.0 |
| 26 | 2.95 | t-butyl bromide | 3.98 | 2.32 | 602.0 |
| 27 | 2.95 | 1,2-di-bromo-2-methylpropane | 3.1 | 2.29 | 94.9 |
| 28 | 2.95 | 2-bromobutane | 3.94 | 2.08 | 164.4 |
| 29 | 1.96 | 2-bromobutane | 2.62 | 1.39 | 77.7 |
| 30 | 2.95 | 2-bromopentane | 4.34 | 2.30 | 230.5 |
| 31 | 2.1 | 2-bromopentane | 3.1 | 1.64 | 87.9 |
| 32 | 2.95 | 2-bromopropane | 3.53 | 2.28 | 125.3 |
| 33 | 1.96 | 2-bromopropane | 2.35 | 1.52 | 67.2 |
| 34 | 2.95 | 3-bromopentane | 4.34 | 2.30 | 288.5 |
| 35 | 2.95 | allyl bromide | 3.51 | 2.32 | 124.0 |

Runs 1-6 correspond to exemplary embodiments of the present invention. In comparing equivalent amounts of bromide from Run 6 to Runs 7, 8, 9, 24, 25, 27, 30, 32, 34 and 36, Run 6 surprisingly and unexpectedly produced a viscosity that was greater than any of the viscosities using other alkyl halides in Runs 7, 8, 9, 24, 25, 27, 30, 32, 34 and 36. Thus, 1-halo-2-methylpropanes, such as 1-bromo-2-methyl-propane (IBB), require less catalyst, in terms of halide level, to produce a polymer having a viscosity of at least 100 cSt than the other alkyl halides.

### Example 2

150 g crude polydecene material produced with trialkylaluminum/isobutylbromide catalyst containing 0.086% (860 ppm) aluminum and 2.24% bromine was diluted with 50 g of decene and treated with 5 g CaO (20 mesh) in a beaker with a magnetic stirrer at 50° C. for 15 minutes. The crude material was then filtered through a 10 micron asbestos pressure filter using 20 to 80 psi nitrogen pressure. The rate of filtration was 10 L/m²/min. The level of aluminum and bromine in the polydecene material after filtration was reduced to 5 ppm aluminum and 0.27% bromine. The filtered CaO contained 1.71% Al. The amount of aluminum and bromine removed was 99.42% and 89.3%, respectively.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for synthesizing polyalphaolefin, comprising:
polymerizing an alpha olefin monomer in the presence of a catalyst system under polymerization conditions, wherein the catalyst system comprises a Group 13 metal catalyst and a 1-halo-2-methylpropane.

2. The process of claim 1, wherein the 1-halo-2-methylpropane is selected from the group consisting of 1-chloro-2-methylpropane, 1-bromo-2-methylpropane, or 1-iodo-2-methylpropane.

3. The process of claim 1, wherein the Group 13 metal catalyst is an alkyl-aluminum compound.

4. The process of claim 1, wherein the Group 13 metal catalyst is selected from the group consisting of trimethylaluminum, triethylaluminum, diethyl(propyl)aluminum, diethyl(butyl)aluminum, ethyl(dipropyl)aluminum, ethyl(dibutyl)aluminum tripropylaluminum, triisopropylaluminum, and tributylaluminum.

5. The process of claim 1, wherein the molar ratio of metal in the Group 13 metal catalyst to halide in the alkyl halide is from 1:2 to 1:16.

6. The process of claim 1, wherein the concentration of Group 13 metal catalyst present during polymerization is from 0.1 to 4.0 wt %, based on the total weight of reactants.

7. The process of claim 1, wherein the concentration of alkyl halide present during polymerization is from 0.5 to 6.0 wt %, based on the total weight of reactants.

8. The process of claim 1, wherein the alpha olefin monomer is 1-decene.

9. The process of claim 1, further comprising the step of reducing from the polyalphaolefin a residual level of the co-catalyst system used to form the polyalphaolefin, by
contacting the polyalphaolefin with a treatment comprising a solid adsorbent selected from the group consisting of an oxide or hydroxide of magnesium, calcium, strontium, barium, sodium and potassium.

10. The process of claim 9, wherein contacting the polyalphaolefin with a treatment comprising a solid adsorbent is done on a fixed bed.

11. The process of claim 9, further comprising filtering the polyalphaolefin to remove a metal of the co-catalyst system.

12. The process of claim 9, wherein the solid adsorbent is calcium oxide.

13. The process of claim 9, further comprising adding a diluent to the polyalphaolefin.

14. The process of claim 9, wherein residual metal is present in the polyalphaolefin in an amount less than 10 ppm.

15. The process of claim 9, wherein filtering the polyalphaolefin further removes a halide of the catalyst system.

16. The process of claim 13, wherein residual halide is present in the polyalphaolefin in an amount less than 3000 ppm.

17. The process of claim 1, wherein the polyalphaolefin has a kinematic viscosity @ 100° C. greater than 90 cSt.

18. The process of claim 1, wherein the polyalphaolefin has a kinematic viscosity @ 100° C. from 90 to 2000 cSt.
